(19) United States
(12) Patent Application Publication
(10) Pub. No.: US 2025/0125014 A1
(43) Pub. Date: Apr. 17, 2025

(54) METHOD AND SYSTEM FOR DECONVOLUTION OF BULK RNA-SEQUENCING DATA

(71) Applicant: NEC Laboratories Europe GmbH, Heidelberg (DE)

(72) Inventors: Francesco ALESIANI, Heidelberg (DE); Giampaolo PILEGGI, Heidelberg (DE); Shujian YU, Heidelberg (DE)

(21) Appl. No.: 18/685,242

(22) PCT Filed: Mar. 10, 2022
(86) PCT No.: PCT/EP2022/056221

§ 371 Date: Feb. 21, 2024

(30) Foreign Application Priority Data
Aug. 27, 2021 (EP) 21193630.7

(51) Int. Cl. G16B 40/20; G06N 3/0455; G06N 3/0464; G06N 3/088; G06N 20/00; G16B 30/00

(52) U.S. Cl. CPC G16B 40/20; G06N 3/0455; G06N 3/0464; G06N 3/088; G06N 20/00; G16B 30/00

(57) ABSTRACT

A method for deconvolution of bulk RNA sequencing data is provided. Input comprising single-cell RNA sequencing (RNA-seq) data is obtained, and diverse datasets are generated based on a principle of same generating mixture probability such that each of the diverse datasets has a same cell type mixture proportion. The generated diverse datasets are used as input datasets for training a prediction model using machine learning, including creating a causal prediction model in which virtual samples are generated from the generated diverse datasets, and performing contrastive learning on the causal prediction model, wherein a contrastive loss is used for the learning of invariant features with respect to a measurement mechanism by which the RNA-seq datasets have been generated. The trained prediction model is used to predict the mixture of cell type quantities in the bulk RNA sequencing data. It can also contribute to predictive optimization regarding patient-specific risks.

### CROSS REFERENCE TO RELATED APPLICATIONS

This application is a U.S. National Phase application under 35 U.S.C. § 371 of International Application No. PCT/EP2022/056221, filed on Mar. 10, 2022, and claims benefit to European Patent Application No. EP 21193630.7, filed on Aug. 27, 2021. The International Application was published in English on Mar. 2, 2023 as WO 2023/025419 A1 under PCT Article 21(2).

### FIELD

The present invention relates to a computer-implemented method and a processing system for deconvolution of bulk RNA-sequencing data.

### BACKGROUND

In the recent years, Next Generation Sequences (NGS) has allowed to obtain information on the genetic activity of the human cells, thus allowing characterizing the Tumor Micro Environment (TME) of patients. With NGS techniques, it is possible to obtain bulk sequencing, that is, a measurement expression level of selected genes in patients. However, bulk sequencing only measures averaged expression levels, due to the bulk mixture containing different cell types, thus creating confounding factors during the characterization of the TME. For this reason, single-cell sequencing (in particular singleRNA-cell sequencing), which analyzes the gene expression at the resolution of single cells of a patient, is becoming more and more widespread. However, single cell sequencing suffers from different problems, such as the high number of dropouts, and higher costs.

Bulk sequencing remains today a more viable solution for obtaining information on gene expressions, but the problem of deducing the original proportion of the different cells in the mixture persists. In order to overcome this limitation deconvolution algorithms have been developed: given a N×M matrix, in which N are the observations, i.e. patients, and M are the genes, deconvolution algorithms output a N×P matrix, in which P are the different cell types, and values of P are the percentage of that cell type in each of the N patients. Current methods rely on using single cell sequencing for different cell types to train predictive algorithms (both probabilistic and ML-based).

US 2021/0142867 A1 discloses a method for deconvolving bulk RNA sequencing data using single-cell RNA-seq of cell types that are relevant to the bulk tissues which are further used to stratify patients. The method comprises selecting a subset of the matrix of counts-based sequencing data from single RNA-seq data. Further, the method involves selecting informative genes, which include using a gradient function whose computation involves discrete approximation. The gene selection method involves excluding non-informative genes that introduce noise. A loss function is defined between the bulk distribution and the mixed single-cell distribution to estimate cell type proportions in the bulk RNA-sequencing data.

### SUMMARY

In an embodiment, the present disclosure provides a computer-implemented method for deconvolution of bulk RNA sequencing data. The method comprises: obtaining input from sources, wherein the input comprises single-cell RNA sequencing (RNA-seq) data; generating, from the single-cell RNA sequencing data, diverse datasets based on a principle of same generating mixture probability such that each of the diverse datasets has a same cell type mixture proportion; using the generated diverse datasets as input datasets for training a prediction model using machine learning, wherein the training comprises: creating a causal prediction model in which virtual samples are generated from the generated diverse datasets; and performing contrastive learning on the causal prediction model, wherein a contrastive loss is used for the learning of invariant features with respect to a measurement mechanism by which the single-cell RNA sequencing datasets have been generated; and using the trained prediction model to predict the mixture of cell type quantities in the bulk RNA sequencing data.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating basic system input and output in a bulk gene deconvolution system according to an embodiment of the present invention;

FIG. 2 is a schematic view illustrating model training and use in a bulk gene deconvolution system according to an embodiment of the present invention;

FIG. 3 is a schematic view illustrating a discrete gate structure implemented in a bulk gene deconvolution system according to an embodiment of the present invention;

FIG. 4 is a schematic view illustrating an auto encoder structure with a discrete gate implemented in a bulk gene deconvolution system according to an embodiment of the present invention;

FIG. 5 is a schematic view illustrating discrete variation features with a discrete gate as part of an encoder implemented in a bulk gene deconvolution system according to an embodiment of the present invention;

FIG. 6 is a schematic view illustrating a causal model for contrastive learning with mixing regression tasks according to an embodiment of the present invention;

FIG. 7 is a schematic view illustrating an approach of contrastive learning with mixing regression tasks according to an embodiment of the present invention;

FIG. 8 is a schematic view illustrating a gene graph construction approach according to an embodiment of the present invention;

FIG. 9 is a schematic view illustrating a transformer network for prediction implemented in a bulk gene deconvolution system according to an embodiment of the present invention;

FIG. 10 is a schematic view illustrating an architecture extending the deconvolution and prediction to the gene expression per cell type in accordance with an embodiment of the present invention;

FIG. 11 is a schematic view illustrating an automatic calibration scheme using separated measurements according to an embodiment of the present invention;

FIG. 12 is a schematic view illustrating an automatic calibration scheme using split measurements according to an embodiment of the present invention;

FIG. 13 is a schematic view illustrating a patient stratification scheme based on cell type predictions according to an embodiment of the present invention;

FIG. 14 is a schematic view illustrating a dataset generation scheme according to an embodiment of the present invention;

FIG. 15 is a schematic view illustrating model training according to an embodiment of the present invention;

FIG. 16 is a schematic view illustrating model training with input reconstruction according to an embodiment of the present invention;

FIG. 17 is a schematic view illustrating a dataset generation scheme based on the usage of the same mix probabilities according to an embodiment of the present invention;

FIG. 18 is a schematic view illustrating contrastive loss training with two parallel networks according to an embodiment of the present invention;

FIG. 19 is a schematic view illustrating the prediction of mixture of cell types and diseases according to an embodiment of the present invention; and

FIG. 20 is a schematic view illustrating conditional mixture prediction according to an embodiment of the present invention.

### DETAILED DESCRIPTION

In accordance with an embodiment, the present invention improves and further develops a method and a system of the aforementioned type for deconvolution of bulk RNA-sequencing data in such a way that the predictions become more stable and reliable.

In accordance with another embodiment, the present invention provides a computer-implemented method for deconvolution of bulk RNA sequencing data, the method comprising: obtaining input from sources comprising single-cell RNA sequencing, RNA-seq, data; generating, from the single-cell RNA sequencing data, diverse datasets based on the principle of same generating mixture probability such that each of the datasets has the same cell type mixture proportion; and using the generated datasets as input datasets for training a model using machine learning. The training comprises creating a causal prediction model in which virtual samples are generated from the generated diverse datasets; and performing contrastive learning on the causal prediction model, wherein the contrastive loss is used for the learning of invariant features with respect to the measurement mechanism by which the single-cell RNA sequencing datasets have been generated. The method further comprises using the trained prediction model to predict the mixture of cell type quantities contained in the bulk RNA sequencing data.

Furthermore, in accordance with an embodiment, the present invention provides a system for deconvolution of bulk RNA-sequencing data and by a tangible, non-transitory computer-readable medium as specified in the independent claims.

Embodiments of the present invention address the problem of estimating the fraction of different cell types from bulk gene sequencing. This is a crucial issue since single-cell sequencing is a costly procedure, while bulk gene sequencing is more affordable. Embodiments of the invention propose a method that uses the diversity of training single-cell sequencing datasets and improves performances on existing methods. Embodiments of the proposed method derive the cell type proportions from the (patient) tissue's aggregated gene expression and can be used to evaluate the patient's response of potential treatment (patient stratification).

Embodiments of the present invention provide a method for bulk gene deconvolution, in particular with an application for patient stratification. The method may include creating a model for reconstructing proportional composition of cell types out of averaged expression quantities. An initial step may include receiving a single-cell dataset and generating diverse datasets each from each source based on the principle of the same probability or mixing single-cell datasets. A next step may include training a model using machine learning, wherein the model includes a discrete gate module and a contrastive loss module. The discrete module may be used for removing noisy features and may be based on the information bottleneck (IB) principle, wherein the contrastive loss may comprise a causal model in which virtual samples are generated from a plurality of single-cell sequence datasets. The contrastive loss promotes the learning of invariant features with respect to the measurement. During the last step, the trained model may be used to predict the mixture/portion/fraction of quantities from the bulk data.

According to an embodiment, the deconvolution system comprises a discrete gate module that is used to promote removal of noisy features from the input datasets. In this context, it may be provided that that the information bottleneck (IB) principle is used as loss function to train the discrete gate module.

According to an embodiment, the deconvolution system comprises an auto encoding mechanism that is configured to transform input gene expressions into latent features.

According to an embodiment, it may be provided that the prediction is extended to the gene expression per cell type. This may be accomplished by execution of the steps of (i) generating in the training data also the bulk gene expression per cell type, and (ii) using a loss function that measures the reconstruction error on the single cell type gene expression and on the reconstruction of the gene expression and the mixture of the gene expression per cell type weighted by the cell type proportion.

According to an embodiment, a simulated distribution of cell types may be used to generate a bulk gene expression from the single-cell RNA sequencing data. The gene expressions of the single cell RNA-sequencing data may be combined in proportion to the probability of a specific cell type to generate an aggregated bulk gene expression. Samples of the aggregated bulk gene expression may then be mapped using a gene graph to train a graph neural network, GNN, wherein the graph of the GNN is learned separately, based on the single cell gene expressions. The output of the GNN may be used to predict the mixture of cell type quantities contained in the bulk RNA-sequencing data.

According to an embodiment, it may be provided that connections among genes are determined by means of a transformer network. The transformer network may then be used to predict the cell types and the gene expressions per cell type. This prediction may be performed by (i) computing matrices K,Q,V (related to key, query, value vectors, respectively) for each gene at each layer of the transformer network, and (ii) computing the cell types and the gene expressions per cell type based on a softmax attention mechanism at each layer of the transformer network.

According to an embodiment, the predicted mixture of cell type quantities may be used for patient stratification. In this context, it may be provided that first a cell type x gene expression matrix is generated based on the predicted mixture of cell type quantities. This matrix may then be combined with domain knowledge information and/or with additional patient information and may be embedded by means of a multimodal embedding model. This embedding may be used for making a patient specific risk prediction with respect to diseases of interest.

According to an embodiment, the gene expression per cell type may be used to automatically calibrate the measurements by which the bulk RNA-sequencing data are obtained. This automatic calibration function may be realized by conducting a separate measurement with an external measuring device, preferably a microscopy. In this separate measurement, cell type counting may be performed and the obtained results may be compared with the cell type counts predicted by the trained prediction model. Based on the obtained cell type count differences, the measurements may be automatically calibrated.

According to an alternative embodiment, the automatic calibration function may be implemented by splitting the sample from which the bulk RNA-sequencing data are obtained in two samples and by conducting separate gene expression measurements on each of the two samples. Next, a first prediction model may be generated and trained for the measurement on a first one of the two samples and a second prediction model may be generated and trained for the measurement on the second one of the two samples. Finally, the predictions may be automatically corrected such that the two separate gene expression measurements yield the same results.

There are several ways how to design and further develop the teaching of the present invention in an advantageous way. To this end, it is to be referred to the dependent claims on the one hand and to the following explanation of preferred embodiments of the invention by way of example, illustrated by the figure on the other hand. In connection with the explanation of the preferred embodiments of the invention by the aid of the figure, generally preferred embodiments and further developments of the teaching will be explained. In the drawing

Embodiments of the present invention provide computer-implemented methods and processing systems for bulk gene deconvolution that implement Machine Learning (ML) techniques to predict cell type percentages (or proportions) when testing on a bulk gene RNA-sequencing sample. In this embodiment, the present invention estimates a model that is invariant to the underlying measurement.

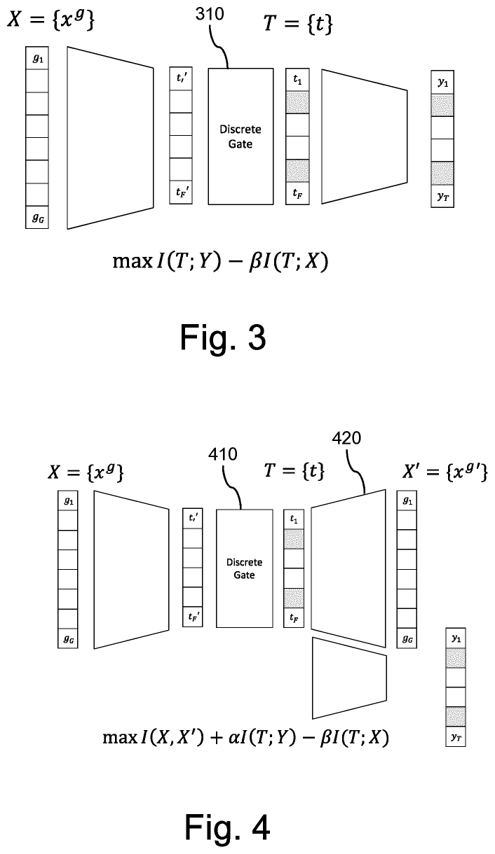

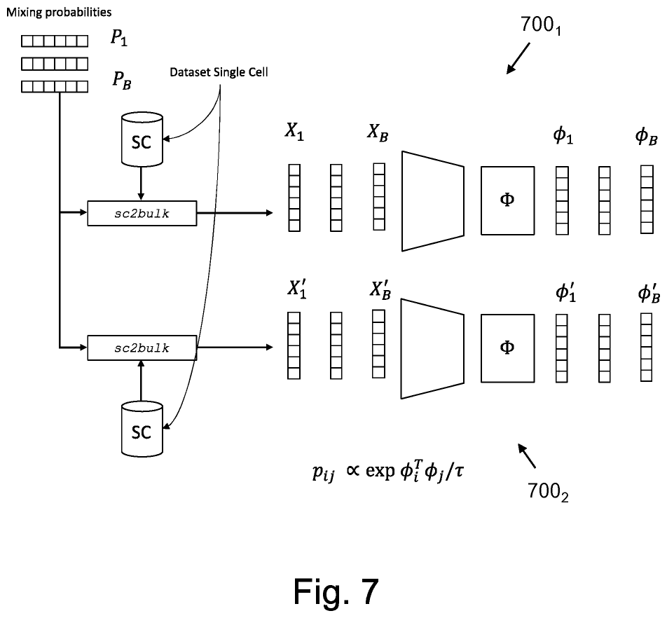

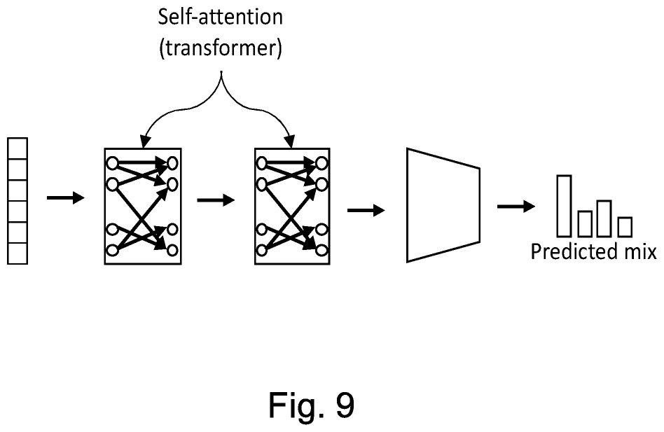

According to an embodiment, the present invention provides a method for creating a model for reconstructing proportional composition of cell types out of averaged expression quantities. The method may comprise the following steps:
1) Receiving the single-cell dataset(s)/measurements;
2) Generating diverse datasets each from each source based on the principle of same probability, i.e. same generating mixture probability (which will be described in more detail in connection with FIG. 7), and/or mixing the single cell datasets (which will be described in more detail in connection with FIG. 4);
3) Creating the model based on the discrete architecture (for example, using the architecture described below in connection with FIG. 3, 4, 5 or 10);
4) Training the network comprising a discrete gate and a contrastive module using the contrastive loss and the information bottleneck (IB) principle (for example, as described in more detail in connection with FIG. 9);
5) Deploying and using the trained model to predict the mixture/portion/fraction of quantities from bulk data.

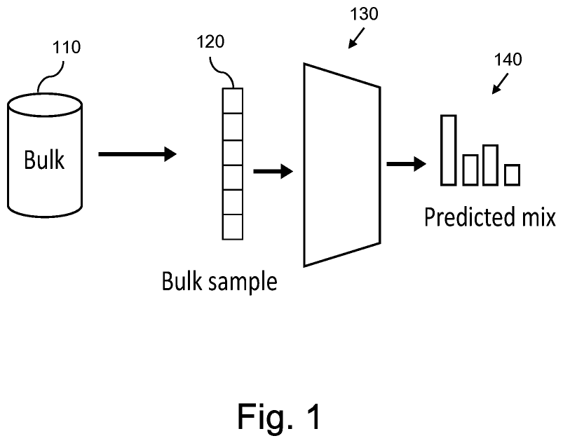

FIG. 1 schematically shows a basic system input and output in a bulk gene deconvolution system according to an embodiment of the present invention. As shown, the system is configured to operate, during the deployment, by getting the bulk RNA-sequencing data 110 (i.e. aggregated sequencing data generated on the basis of different cell types contained in the respective sequenced sample with a given mixture probability), generating bulk samples 120 from the bulk data 110 and, by using a ML technique 130 that will be described in further detail below, predicting the mixture, as shown at 140. For example, in case the bulk data 110 contain gene expression data, the system may be configured to predict the proportion of cells per cell type.

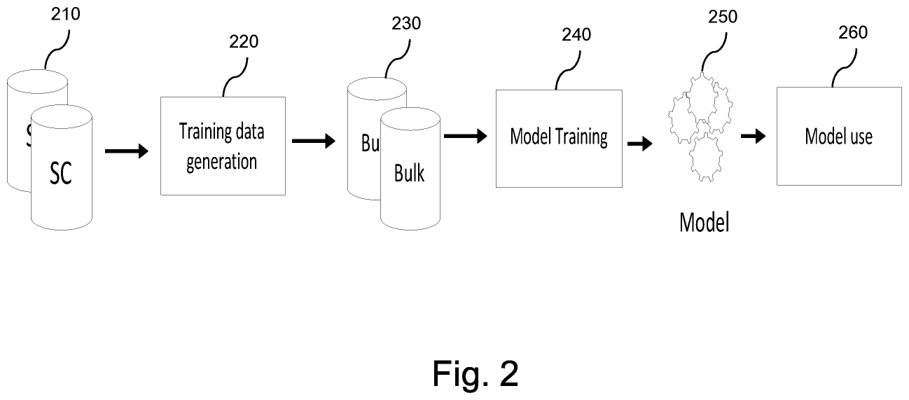

FIG. 2 schematically illustrates model training and use in a bulk gene deconvolution system according to an embodiment of the present invention. The training of the model uses single cell (SC) data 210, which may be SC RNA-seq data generated by measuring the gene expression from a single cell. The data 210 is used by a training data generation component 220 to generate simulated bulk data 230 for training (as shown at 240) a model 250 to predict the input mixture from the simulated bulk data. After training, the model 250 can be used to predict the input mixture of unknown real bulk data, as shown at 260.

In a brief overview, according to embodiments of the invention, the systems and methods for bulk gene deconvolution disclosed herein may implement one or more of the following aspects, each of which will be described in more detail further below:
1) The use of contrastive loss to promote invariant feature learning and generating the samples from coming mixing probabilities;
2) The use of a discrete gate to remove noisy features;
3) The use of hidden discrete masking to promote removal of not invariant features, when dealing with sequential learning;
4) The use of variational Bayesian methods to estimate a bound in the mixture percentage;
5) The use of the Information Bottleneck (IB) approach to remove redundancy or spurious information in features that do not improve prediction; and/or
6) The use of a decoder to reconstruct the input gene bulk.

According to embodiments of the invention, the systems and methods for bulk gene deconvolution disclosed herein may receive one or more of the following data as input:
1) The single cell expression of the genes;
2) Possibly existing training bulk datasets;
3) The possible disease(s); and/or
4) The type of tissue (e.g. the area of the origin).

In an embodiment, the system may receive further information, in particular Electronic Health Record information related to a status of the respective patient, (e.g. vital parameters like heart rate, etc.). This additional information may be associated to the measured RNA-seq data and may be used in an embodiment that uses a particular disease as input and conditions the model on this information.

According to embodiments of the invention, the systems and methods for bulk gene deconvolution disclosed herein may be configured to output the proportions of the different cell types contained in a bulk sequencing probe. In addition, the output may include information on active diseases and on the status of cells.

FIG. 3 schematically illustrates the basic architecture of a bulk gene deconvolution system according to an embodiment of the present invention, including a discrete gating function 310 that is used to filter noisy features. As shown in FIG. 3, X={x^{g}} denotes the measured input, for example the gene expression of a cell or a bulk of cells, i.e. gᵢ denote the respective genes. T′={t′₁, . . . , t′_{F}} are the latent variables. In the illustrated embodiment, the latent variables T′ are not used directly to predict the cell type proportion Y={y₁, . . . , y_{T}}, but first the latent feature is discretized and gated by discrete gate 310 to produce the variables T={t₁, . . . , t_{F}}. The principle is to reduce the number of active latent features based on the Information Bottleneck (IB) approach. The loss function is composed of the mutual information I(T;Y) from the output and the latent and I(T;X) is the mutual information from the input to the latent, this promote the removal of features that are not relevant for the prediction task.

According to an embodiment of the present invention, the bulk gene deconvolution system may be configured to use a discrete gating function 410 as well as an auto encoding mechanism 420, as exemplarily illustrated in FIG. 4. In this architecture with an auto encoder structure 420 with discrete gate 410, the input feature, e.g. gene expression X, is transformed into latent feature T′ and T after the discrete gate 410. The latent features T are also used to reconstruct the input as X′. As depicted in FIG. 4, the training function needs to be modified to include the reconstruction of the input as X′, so now one has a term I(X; X′).

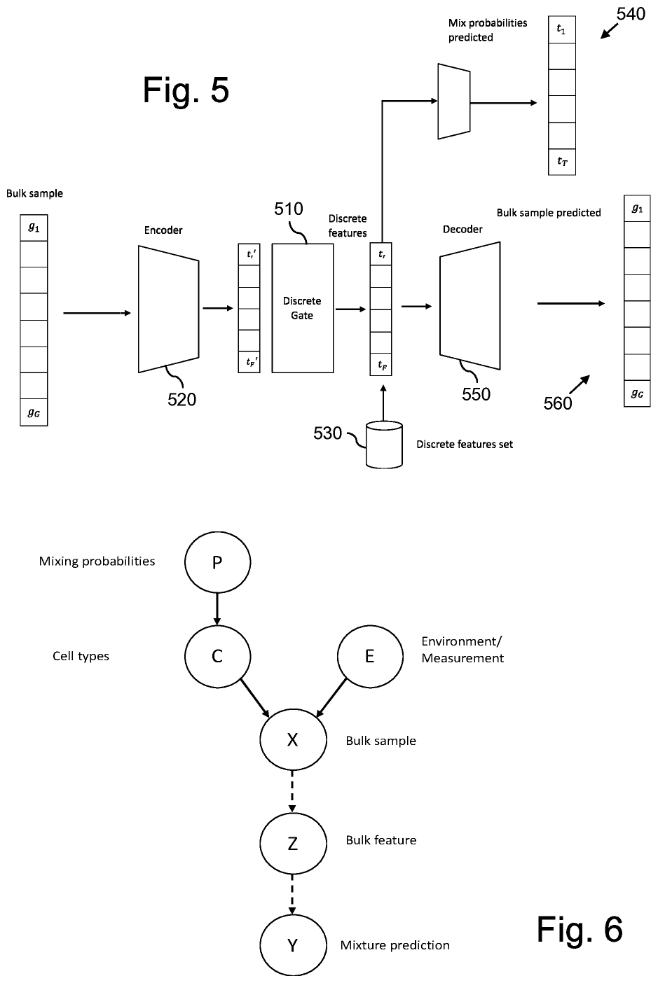

According to an embodiment of the present invention, the bulk gene deconvolution system also considers the case, where the features are discrete (i.e. the latent features t′ are discrete values). In this regard, FIG. 5 exemplarily illustrates discrete variation features, where the discrete gate 510 is part of the encoder 520. This is used to compress the features and to limit the number of features. In this architecture, a lookup table 530 with discrete feature is used to quantize the feature space. This reduces the features to a pre-defined number of features of the latent space. The latent variable T′ is also compressed in dimension by gating to zero some of the values. The latent variable T′ is then used to predict the cell types (as shown at 540 variable), but also—with the help of decoder 550—the input G (as shown at 560).

FIG. 6 schematically illustrates a causal model for the bulk sample generation and prediction in accordance with an embodiment of the present invention. The causal model may be used for contrastive learning with mixing regression task. In this context and as illustrated in FIG. 6, it may be provided that the mix prediction Y is generated by bulk features Z, which depend on the bulk sample observations X. IN the illustrated embodiment, the observations X are assumed to depend on two inputs, the Environment E and the cell expression C. The bulk cell expression C depends on the mixture of the cells present in the sample on which the measurements were carried out.

For learning an invariant model, i.e. a model that is invariant to the multiple single cell sequencing environments, embodiments of the invention provide a sample generation process that starts with sample mixing probabilities and, then, generates the bulk samples according to these probabilities. The invariant model may then be learned using the contrastive loss. The contrastive loss may require that the feature probabilities (pᵢⱼ) computed from the mapping of the features (ϕᵢ, ϕ′ᵢ) using a softmax function (or normalized exponential function) pᵢⱼ∝exp ϕᵢ^{T}ϕⱼ/τ are similar on two set of samples taken from two different single cell datasets:

$${p}_{ij} = \frac{exp \frac{{ϕ}_{i}^{T} {ϕ}_{j}}{τ}}{{\sum }_{k} exp \frac{{ϕ}_{i}^{T} {ϕ}_{k}}{τ}}$$

The contrastive learning is described in more detail in connection with FIG. 7, which schematically illustrates an embodiment of contrastive learning with mixing regression tasks. According to this approach, two parallel equal networks 7001, 7002 are used to learn the mixture of probability. The bulk is generated from an input mixture P1, P2 from the single cell. These two sets of sample have the same percentage of cells. Therefore, the two networks 7001, 7002 can be trained using the constative loss, where the feature of corresponding probabilities mixture is asked to be closer than other mixture. This loss function may be added to the other reconstruction losses.

As regards the deployed loss functions, according to an embodiment, it may be provided that reconstruction loss (11, 12, root mean square) is used for the mixing probabilities and when predicting the gene expression, while the KL divergence may be considered as loss function in the contrastive learning described above. The information bottleneck loss may be used to reduce the information at the feature level.

Next, the case is considered, where the network encoding and predicting is a graph convolution network. In this case, the gene expression may be converted into a graph, where the connections are defined using the k-nearest neighbor based on a similarity between two gene activation patterns.

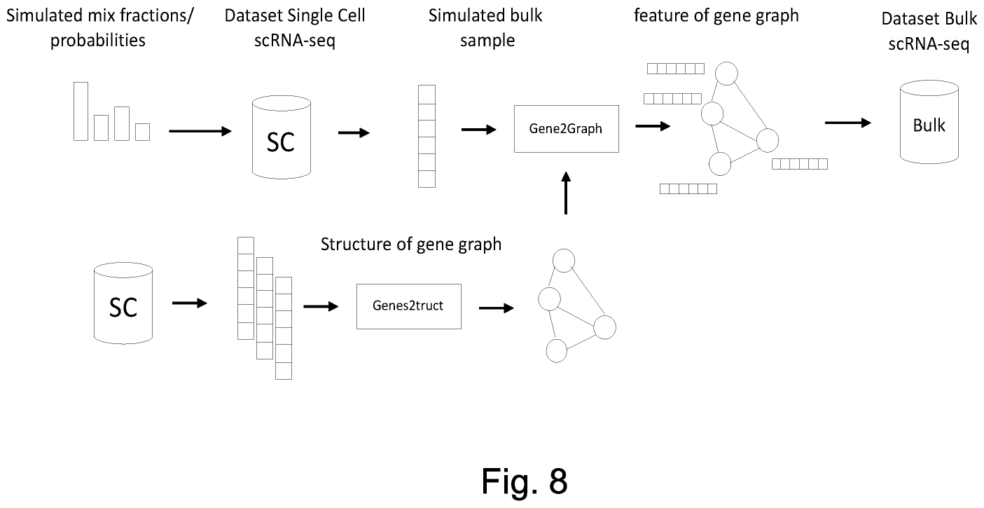

In this context, FIG. 8 schematically illustrates a gene graph construction approach in accordance with an embodiment of the present invention. As shown, according to this approach a simulated distribution of the cell types is used to generate the bulk gene expression from a database of single cell expressions. Indeed, each cell sample of a specific type in the database has its own gene expression. These gene expressions are combine in proportion to the probability of a specific cell type to generate the aggregated gene expression (bulk gene expression). These samples are then mapped using a gene graph to train a graph neural network (GNN). The graph of the GNN may be learned separately, based on the single cell gene expressions. This operation can be repeated end-to-end to refine the gene graph. The output of the GNN can be used to predict the cell type mixture.

FIG. 9 schematically illustrates an embodiment of the present invention where, instead of the neural network, a transformer network is used for prediction, i.e. the network layers are transformer modules. In this case the connection among genes is determined by the transform network, and the transformer network can then be used to predict the cell type and possibly also the gene expression per cell type. In this context it may provided that K,Q,V (learned matrices related to the key, query, value vectors, respectively) are computed for each gene at each layer and the output is computed based on the attention mechanism A=softmax(Q^{l}K^{lT}), X^{l+1}=X^{l}+BN(A^{l}X^{l}) at each layer.

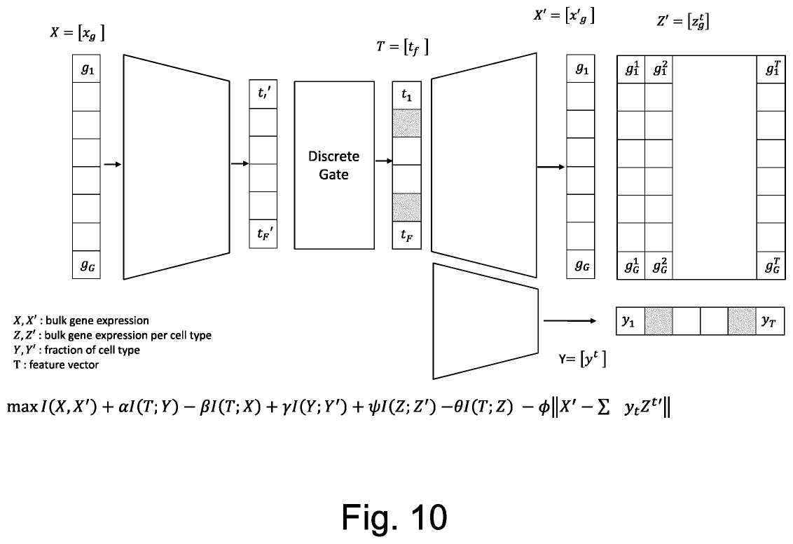

FIG. 10 schematically illustrates an architecture in accordance with an embodiment of the present invention that extends the deconvolution and prediction to the gene expression per cell type, similar to the embodiment described above in connection with FIG. 4. In the case shown in FIG. 10, however, the architecture is configured to generate in the training data also the bulk per cell type. Furthermore, a loss function is added that measures the reconstruction error on the single cell type expression and on the reconstruction of the gene expression and the mixture of the gene expression per cell type weighted by the cell type proportion. As shown in FIG. 10, the latent discrete and gated features (T) are used to predict the gene expression per cell type (Z′), along with the cell type mixture (Y). The loss function now has terms that measure the reconstruction error not only of the mixture Y, the total gene expression X′, but also the per cell type expression Z′.

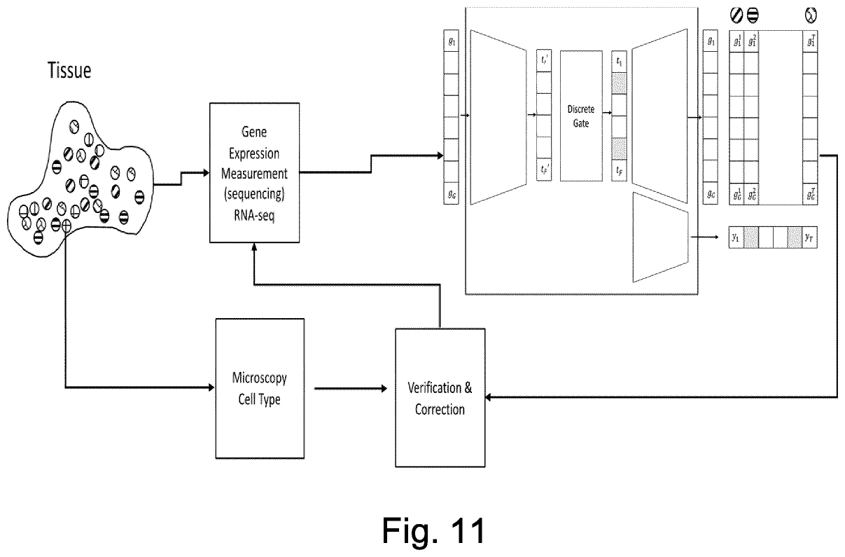

Embodiments of the present invention address the problem of improving the RNA-sequencing measurements performed in laboratory based on the prediction of the cell type and gene expression per cell type. In this context, two different scenarios are considered where the output of the proposed bulk gene deconvolution system is used to calibrate a sequencing system. When there is another way to evaluate the cell type composition, for example using a microscopy, the measurement system can be modified so that the two quantities become close. This scenario including an automatic calibration using separated measurements is schematically illustrated in FIG. 11. In this architecture, the gene expression per cell type is used to automatically calibrate the measures. A separate measure is used coming from, e.g., a microscopy where the cell types are counted. This information is then combined with the cell types predicted by the model and used to correct the measurements.

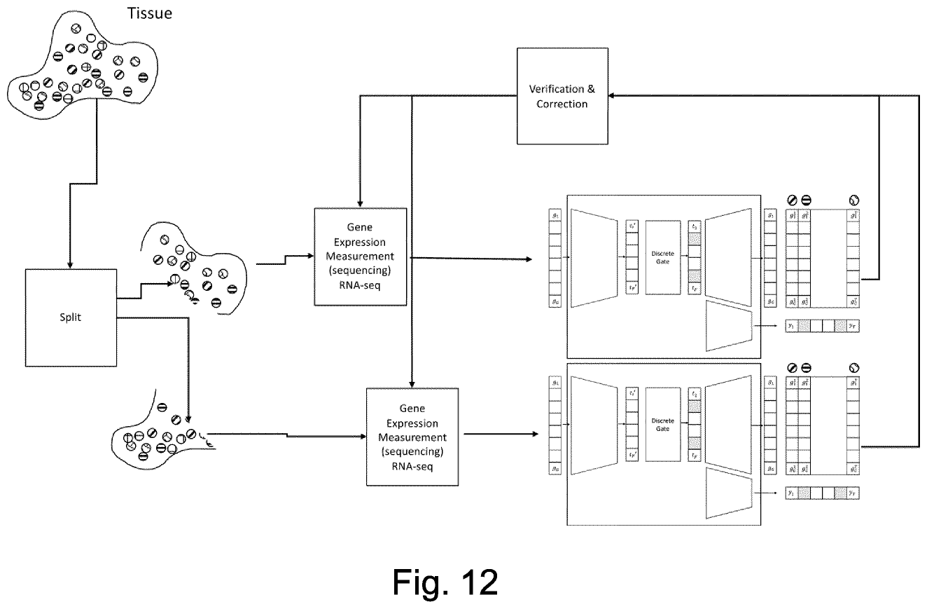

Alternatively, as exemplary shown in FIG. 12, the sample tissue may be split in two samples, which may then be measured separately, for example sequentially or using separate measurement systems. As shown in FIG. 12, for the two measurements two parallel pipelines may be deployed, each pipeline having its own prediction model. This architecture allows to automatically calibrate the measurements by asking the two pipeline to be coherent. To this end, the predictions of the two models may be automatically corrected such that the measurements from the two pipelines are the same or are at least close. In other words, the outputs of the two separate measurements are compared with each other and, based on the comparison, the measurement system(s) are calibrated such that the measurements are the same or at least close.

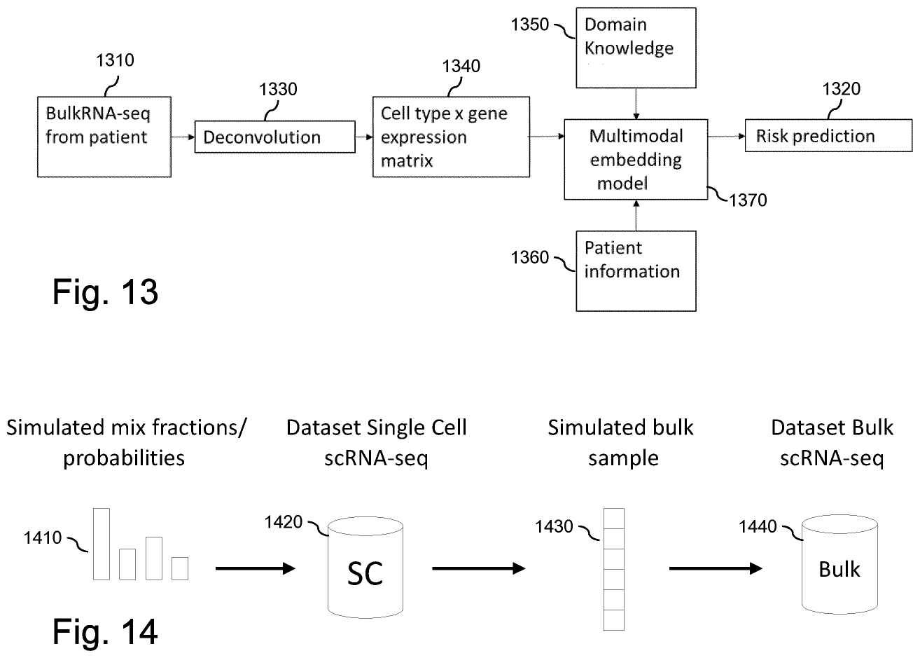

Embodiments of the present invention provide methods and systems that use bulk to mixture predictions as disclosed herein for the purpose of patient stratification. A respective patient stratification workflow is exemplarily illustrated in FIG. 13. Basically, the workflow aims at, given as input a bulk of cells from a patient or, more specifically, bulk RNA-sequencing data 1310 of a patient, producing a risk probability 1320 for the patient to develop a certain disease. According to an embodiment, the bulk RNA-sequencing data 1310 is used as input for a deconvolution system 1330 configured to perform deconvolution according to the methods described in the present disclosure. The output of the deconvolution system 1330, namely a cell type x gene expression matrix 1340, may be linearly combined with domain knowledge information 1350 (e.g., gene annotations) and/or additional patient information 1360 (e.g., Electronic Health Records, EHR), and embedded through a multimodal embedding model 1370 (e.g., GNN, autoencoder, etc.). The embedding can be given to downstream model that is configured to predict the risk for the patient to develop the disease of interest. Specifically, the risk can be determined based on information extracted from the genetic profiles of the single cell types.

FIG. 14 is a schematic view illustrating a dataset generation scheme according to an embodiment of the present invention. The generation starts with a mixture of probabilities 1410 and uses a single-cell (SC) gene expression dataset 1420 to generate simulated bulk samples 1430, which are then aggregated to a (bulk) gene expression dataset 1440.

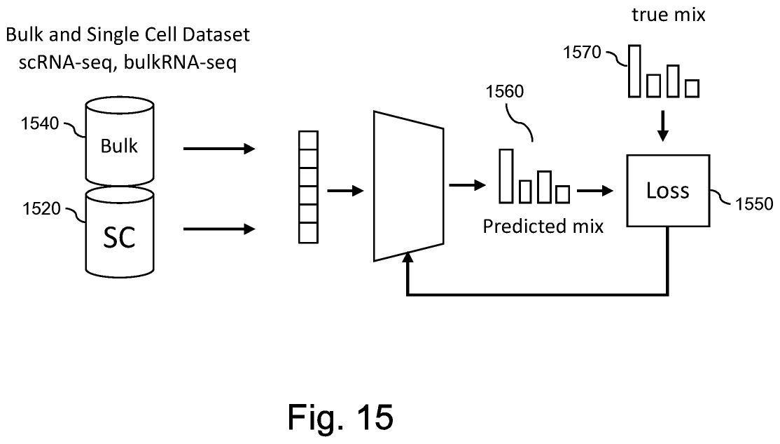

FIG. 15 is a schematic view illustrating model training according to an embodiment of the present invention. During the model training, a single cell (SC) gene expression dataset 1520 is used as additional input along with an (aggregated) bulk gene expression dataset 1540 (e.g., generated as described above in connection with FIG. 14). The loss function 1550 measures the difference between the predicted mixture 1560 and the true mixture 1570. As already explained above in connection with FIG. 1, once the model is trained and when using the trained model, the model input is the bulk data and the output is the predicted mixture of cell types.

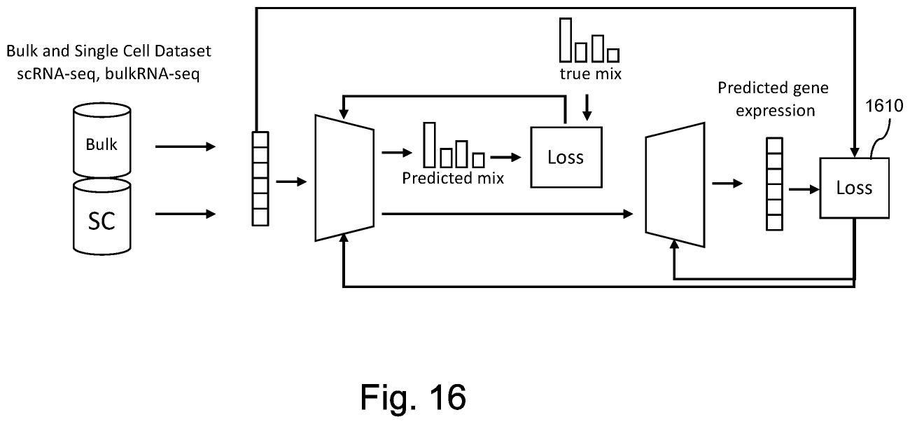

When using a decoder network, the training may be performed in the modified way. In this context, FIG. 16 provides a schematic view illustrating model training with input reconstruction according to an embodiment of the present invention. Here, in addition to predicting the mixture of the cell types, the model is further utilized to also reconstruct the input data and use an additional loss 1610. When using the contrastive loss the training may be changed accordingly.

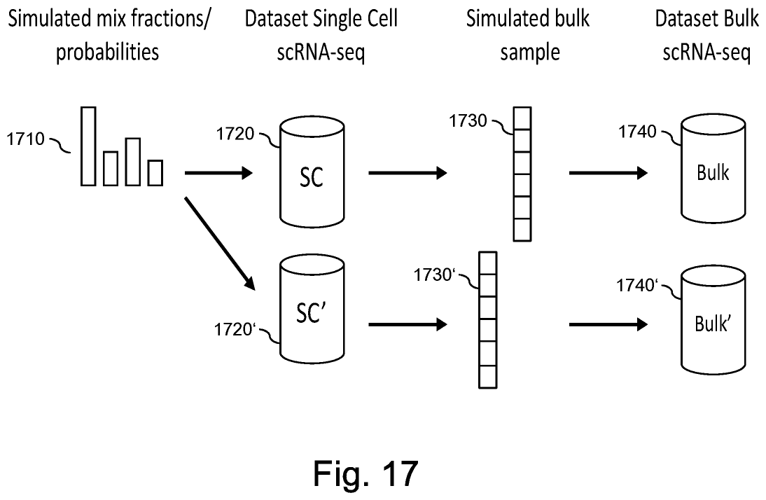

FIG. 17 is a schematic view illustrating a dataset generation scheme based on the usage of the same mix probabilities according to an embodiment of the present invention. As shown, the data generation starts from the same mixture 1710, but the single cell gene expressions 1730, 1730′ are taken from two different SC databases 1720, 1720′, such that two separate bulk scRNA-seq datasets 1740, 1740′ are generated.

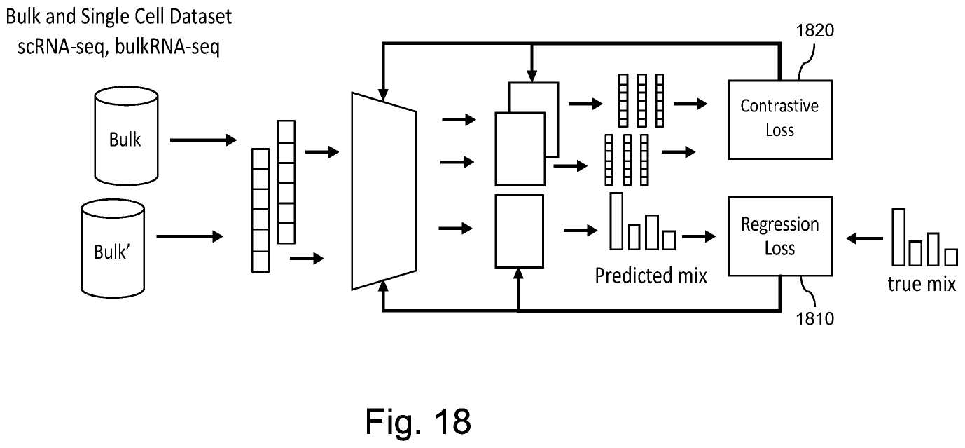

FIG. 18 is a schematic view illustrating a modified training scheme according to an embodiment of the present invention that is used when training with the contrastive loss. In this case, the training is performed with two parallel networks, and there are two different loss types: first, a reconstruction loss 1810 on the mixture of cell types and, second, a contrastive loss 1820 that forces to have similar probability distributions for samples generated by the same mixture.

According to embodiments, the present invention provides methods and systems for conditional prediction of cell type on diseases. These embodiments consider the case where information on the disease is available when training the regression model. In this case, the virtual bulk can be used and the disease mixture can be added. When estimating, also the presence of diseases can be predicted.

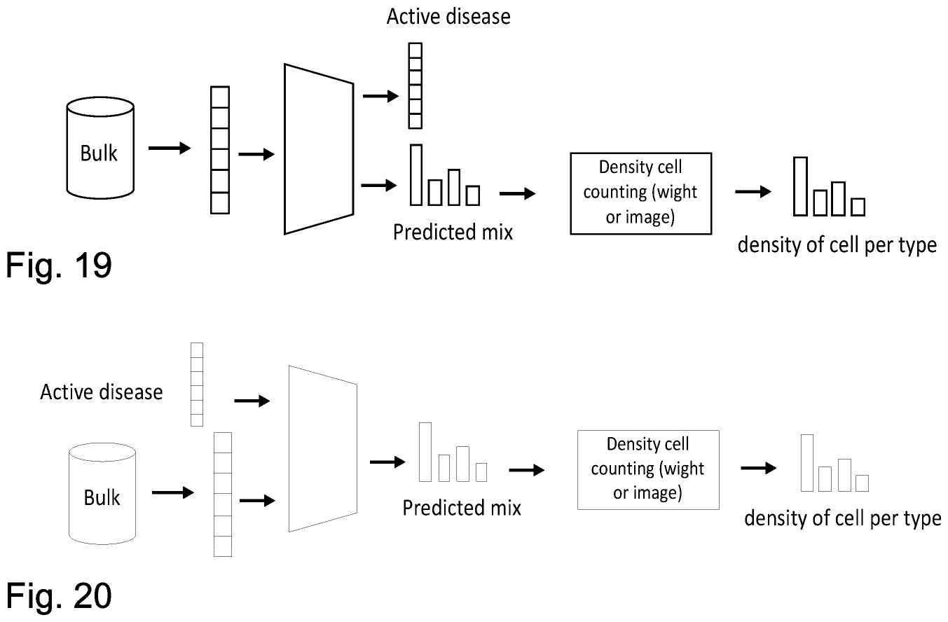

FIG. 19 is a schematic view illustrating the prediction of mixture of cell types and diseases according to an embodiment of the present invention. In addition to predict the mixture of cell type, the prediction scheme adds the information of counting or density estimation from the image to derive the density or counting of the cell types. In an embodiment, the network is also asked to predict the disease that is present in a specific bulk genetic expression. During training, the disease information is an output and a loss is added that measure the reconstruction loss of the prediction of the disease.

Alternatively, when training for single diseases separately, in this case, the disease may be used as input (e.g. using an indicator vector or hot encoded) and the model is conditioned on this information. In this way, one can have different predictions depending on the hypothesis on the disease.

FIG. 20 is a schematic view illustrating conditional mixture prediction according to an embodiment of the present invention. In this embodiment, additional information on the disease related to a particular cell type is used. If this information is available, or at least reasonably true, the model, which may be implemented in form of a conditional neural network, will provide a prediction, which depends on the presence of a specific disease. During training, this information is used as input.

The information of the predicted disease can be used to reject the prediction or to select a dedicated model specific for the specific disease. In addition to the disease, other information (for example from the Electronic Health Record information) can be used similarly.

Embodiments of the present invention provide the following advantages: By modelling the bulk as different environments, the model can learn invariant and more stable predictions. The method thus provides more reliable information. An additional confidence information may be provided when training with variational methods. With the contrastive loss, the trained feature can be used for further processing, if the granularity is at the level of cell type. The prediction method according to embodiments of the present invention as disclosed herein outperforms in almost all scenarios the competing Scaden method (for reference, see Menden, K., Marouf, M., Oller, S., Dalmia, A., Magruder, D. S., Kloiber, K., Heutink, P. and Bonn, S., 2020. Deep learning-based cell composition analysis from tissue expression profiles. Science advances, 6(30), p.eaba2619).

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A computer-implemented method for deconvolution of bulk RNA sequencing data, the method comprising:
obtaining input from sources, wherein the input comprises single-cell RNA sequencing RNA-seq data;
generating, from the single-cell RNA sequencing data, diverse datasets based on a principle of same generating mixture probability such that each of the diverse datasets has a same cell type mixture proportion;
using the generated diverse datasets as input datasets for training a prediction model using machine learning, wherein the training comprises:
creating a causal prediction model in which virtual samples are generated from the generated diverse datasets; and
performing contrastive learning on the causal prediction model, wherein a contrastive loss is used for the learning of invariant features with respect to a measurement mechanism by which the single-cell RNA sequencing datasets have been generated; and
using the trained prediction model to predict the mixture of cell type quantities in the bulk RNA sequencing data.

2: The method according to claim 1, further comprising:
creating a discrete gate module and using the discrete gate module for the removal of noisy features from the input datasets.

3: The method according to claim 2, further comprising:
training the discrete gate module by using an information bottleneck principle as a loss function.

4: The method according to claim 1, further comprising:
transforming, by an auto encoding mechanism, input gene expressions into latent features.

5: The method according to claim 1, further comprising extending the prediction to gene expression per cell type, comprising the steps of:
generating in a training data a bulk gene expression per cell type;
using a loss function that measures a reconstruction error on a single cell type gene expression and on a reconstruction of a gene expression and a mixture of the gene expression per cell type weighted by a cell type proportion.

6: The method according to claim 1, further comprising:
using a simulated distribution of cell types to generate a bulk gene expression from the single-cell RNA sequencing data;
combining gene expressions of the single-cell RNA sequencing data in proportion to a probability of a specific cell type to generate an aggregated bulk gene expression;
mapping samples of the aggregated bulk gene expression using a gene graph to train a graph neural network (GNN), wherein the graph of the GNN is learned separately, based on single cell gene expressions; and
using an output of the GNN to predict the mixture of cell type quantities in the bulk RNA sequencing data.

7: The method according to claim 1, further comprising:
determining connections among genes using a transformer network; and
using the transformer network to predict the cell types and gene expressions per cell type by performing the steps of:
computing matrices K,Q,V related to key, query, value vectors, respectively, for each gene at each layer of the transformer network, and
computing the cell types and the gene expressions per cell type based on a softmax attention mechanism at each layer of the transformer network.

8: The method according to claim 1, further comprising:
using the predicted mixture of cell type quantities for patient stratification.

9: The method according to claim 8, wherein patient stratification comprises:
generating, based on the predicted mixture of cell type quantities, a cell type x gene expression matrix;
combining the generated matrix with domain knowledge information and/or with additional patient information;
embedding the generated matrix using a multimodal embedding model; and
using the embedding for making a patient specific risk prediction with respect to diseases of interest.

10: The method according to claim 1, further comprising using gene expression per cell type to automatically calibrate measurements by which the bulk RNA sequencing data are obtained, comprising the steps of:
conducting a separate measurement with an external measuring device;
performing cell type counting in the separate measurement and comparing the obtained results with cell type counts predicted by the trained prediction model; and
calibrating the measurements based on the comparison.

11: The method according to claim 1, further comprising using gene expression per cell type to automatically calibrate the measurements by which the bulk RNA sequencing data are obtained, comprising the steps of:
splitting a sample from which the bulk RNA sequencing data is obtained in two samples and conducting separate gene expression measurements on each of the two samples;
generating and training a first prediction model for a measurement on a first one of the two samples and generating and training a second prediction model for a measurement on the second one of the two samples; and
automatically correcting the predictions such that the two separate gene expression measurements yield the same results.

12: A system for deconvolution of bulk RNA sequencing data, the system comprising one or more processes that, alone or in combination, are configured to provide for the execution of the following steps:
obtaining input from sources, wherein the input comprises single-cell RNA sequencing RNA-seq data;
generating, from the single-cell RNA sequencing data, diverse datasets based on a principle of same generating mixture probability such that each of the diverse datasets has a same cell type mixture proportion;
using the generated datasets as input datasets for training a prediction model using machine learning, wherein the training comprises:
creating a causal prediction model in which virtual samples are generated from the generated diverse datasets; and
performing contrastive learning on the causal prediction model, wherein the contrastive loss is used for the learning of invariant features with respect to a measurement mechanism by which the single-cell RNA sequencing datasets have been generated; and
using the trained prediction model to predict the mixture of cell type quantities in the bulk RNA sequencing data.

13: The system according to claim 12, further comprising:
a discrete gate module trained by using an information bottleneck principle as a loss function to remove noisy features from the input datasets, and/or
an auto encoding mechanism configured to transform input gene expressions into latent features.

14: The system according to claim 12, further comprising a patient stratification component configured to:
generate, based on the predicted mixture of cell type quantities, a cell type x gene expression matrix;
combine the generated matrix with domain knowledge information and/or with additional patient information;
embed the generated matrix using of a multimodal embedding model; and
use the embedding for making a patient specific risk prediction with respect to diseases of interest.

15: A tangible, non-transitory computer-readable medium having instructions thereon which, upon being executed by one or more processors, alone or in combination, provide for execution of a method for deconvolution of bulk RNA sequencing data, the method comprising:
obtaining input from sources wherein the input comprises single-cell RNA sequencing RNA-seq data;
generating, from the single-cell RNA sequencing data, diverse datasets based on a principle of same generating mixture probability such that each of the datasets has a same cell type mixture proportion;
using the generated diverse datasets as input datasets for training a prediction model using machine learning, wherein the training comprises:
creating a causal prediction model in which virtual samples are generated from the generated diverse datasets; and
performing contrastive learning on the causal prediction model, wherein a contrastive loss is used for the learning of invariant features with respect to a measurement mechanism by which the single-cell RNA sequencing datasets have been generated; and
using the trained prediction model to predict the mixture of cell type quantities in the bulk RNA sequencing data.

16: The method according to claim 10, wherein the external measuring device is a microscope.
